(19) United States
(12) Patent Application Publication
(10) Pub. No.: US 2026/0317377 A1
(43) Pub. Date: Oct. 15, 2026

(54) VERAGUAMIDE COMPOUNDS

(71) Applicants: University of Kentucky Research Foundation, Lexington, KY (US); Board of Regents, The University of Texas System, Austin, TX (US)

(72) Inventors: Kevin J. Tidgewell, Lexington, KY (US); Benedict Kolber, Dallas, TX (US); Chhabi Chaudhary, Lexington, KY (US)

(21) Appl. No.: 19/634,568

(22) Filed: Mar. 31, 2026

Related U.S. Application Data
(60) Provisional application No. 63/781,183, filed on Mar. 31, 2025.

(51) Int. Cl. C07K 11/00; A61K 38/00; A61K 45/06; A61P 25/04; C07K 11/02

(52) U.S. Cl. CPC C07K 11/00; A61K 45/06; A61P 25/04; C07K 11/02; A61K 38/00

(57) ABSTRACT

Disclosed is a veraguamide compound or pharmaceutically acceptable salt thereof, wherein the compound is derived from a veraguamide, and wherein an ester linkage is replaced with an amide linkage.

### CROSS REFERENCE TO RELATED APPLICATION

This application claims priority from U.S. Provisional Application Ser. No. 63/781,183 filed Mar. 31, 2025, the entire disclosure of which is incorporated herein by this reference.

### STATEMENT OF GOVERNMENT INTEREST

This disclosure was made with government support under grant numbers R61NS127271 and 2R15AT008060-02 awarded by the National Institutes of Health. The government has certain rights in the disclosure.

### BACKGROUND

Chronic pain treatment is challenged by opioid dependence, variable patient responses, and a lack of non-opioid alternatives. Chronic pain management lacks effective, long-term solutions, leading to overprescription, addiction, and insufficient relief. There are gaps that need to be filled of developing safer analgesics that will improve patient outcomes.

The veraguamide class of molecules could be a part of this solution. By binding to the sigma-2 receptor and altering store operated calcium entry (SOCE) system function, there is promise in developing a neuropathic pain treatment. Early analogs have shown efficacy in vivo for the reduction of nociception in a mouse pain model.

### BRIEF DESCRIPTION

Disclosed herein is a compound or pharmaceutically acceptable salt thereof, wherein the compound is derived from a veraguamide, wherein an ester linkage is replaced with an amide linkage.

Also disclosed is a pharmaceutical composition comprising the above-referenced compound, a veraguamide natural product, or a combination thereof, and a pharmaceutically acceptable excipient.

A method for treating or preventing pain in a patient in need thereof is disclosed, comprising administering to the patient in need thereof a therapeutically effective amount of the above-referenced compound, a veraguamide natural product, or a combination thereof, or the above-referenced pharmaceutical composition.

A method for inhibiting a nociceptor cell, comprising contacting the cell with an effective amount of the above-referenced compound, a veraguamide natural product, or a combination thereof, or the above-referenced pharmaceutical composition.

A method for inhibiting phosphorylation of eukaryotic initiation factor 2α (eIF2α) is disclosed, comprising contacting the eukaryotic initiation factor 2α with an effective amount of the above-referenced compound, a veraguamide natural product, or a combination thereof, the above-referenced pharmaceutical composition.

### BRIEF DESCRIPTION OF THE DRAWINGS

The following figures are exemplary embodiments.

FIG. 1 is a representation of the chemical structures of veraguamides O (1), P (2), and C (3);

FIG. 2 is a representation of the COSY and TOCSY connectivity and selected HMBC correlations for veraguamide O (1), of Scheme 1 of Example 1;

FIG. 3A a photographic image of sample DUQ0008 of Example before collection 3;

FIG. 3B is a confocal microscopic image of DUQ0008 of Example 1 at 10× magnification with FITC (487 nm), TRITC (560 nm) and Cy5 (637 nm) channels;

FIG. 3C is light microscopic image of DUQ0008 of Example 1 at 10× magnification;

FIG. 3D is a phylogenetic tree of species related to DUQ0008 of Example 1;

FIG. 4A is a graph illustrating the isolated veraguamide activity for cell viability (percent, %, measured in DMSO) versus concentration (micromoles per liter, μM) of veraguamide O and veraguamide C in the estrogen-positive breast cancer cell line, MCF-7, of Example 1;

FIG. 4B is a graph illustrating the isolated veraguamide activity for cell viability (%), versus concentration (μM) of veraguamide O and veraguamide C in the TNBC cell line, MDA-MB-231 of Example 1;

FIG. 4C is a graph illustrating the isolated veraguamide activity for cell viability (%), versus concentration (μM) of veraguamide C in the HEK-293 cell line of Example 1

FIG. 5 is a graph illustrating cell viability (%) versus dose (μM) of a control sample and analogs of Example 2;

FIG. 6 is a graph illustrating change from the 37° C. baseline (%) versus time (hr, hours) of Example 3; and

FIG. 7A to FIG. 7F are graphs illustrating mean gray intensity for varying concentrations of test compounds in comparison to a vehicle of Example 4.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The sigma-2 receptor and the Store-Operated Calcium Entry (SOCE) system are interconnected in several ways, particularly in their roles in regulating intracellular calcium levels and influencing cellular processes such as signaling. While the two systems are distinct in their molecular identity, there is evidence suggesting that the sigma-2 receptor can modulate the SOCE pathway. The sigma-2 receptor and Store-Operated Calcium Entry (SOCE) system both play significant roles in cellular calcium signaling, and their interaction may have important implications for pain management. Although research in this specific area is still emerging, there are some key insights into how these two systems can influence pain perception and response to pain therapies.

Natural products are crucial sources for drug development and discovery. In the last two decades, marine cyanobacteria have emerged as a leading source for novel bioactive natural products. The high degree of chemical diversity among cyanobacterial natural products is attributed to their biosynthetic machinery, which integrates polyketide synthases (PKS) and nonribosomal peptide synthases (NRPS) into mixed biosynthetic pathways resulting in diverse bioactive secondary metabolites. This broad spectrum of marine cyanobacterial natural products often translates into a wide array of biological activities, including, but not limited to, anticancer, antibacterial, antimalarial, anti-inflammatory, and neuroactive activities.

A structurally-related family of natural products (“veraguamides”) were isolated from a collection of cf. Oscillatoria margaritifera obtained from the Coiba National Park, Panama. Veraguamides have previously been shown to have cytotoxic effects in the human lung cancer cell line H-460. In particular, the veraguamides exhibit significant cytotoxicity, primarily through mechanisms such as cell cycle arrest, apoptosis induction, and disruption of microtubule dynamics. These properties make it a promising candidate for the development of anticancer drugs.

While veraguamide depsipeptides have been screened against human lung (H-460), colon (HCT-116), and breast cancer (MCF-7) cell lines, so far, none have been investigated for their cytotoxic effects against the more resistant and aggressive triple-negative breast cancer (TNBC) cells. In addition, although both cyclic and linear veraguamides have been reported, with cyclic versions being more common, the cytotoxic effects of most linear depsipeptides against human cancer cell lines have not been determined. To that end, the inventors hereof isolated and determined the structures of two new linear depsipeptides, veraguamides O (1) and P (2), from a Panamanian marine cyanobacterium, known cyclic veraguamide C (3).

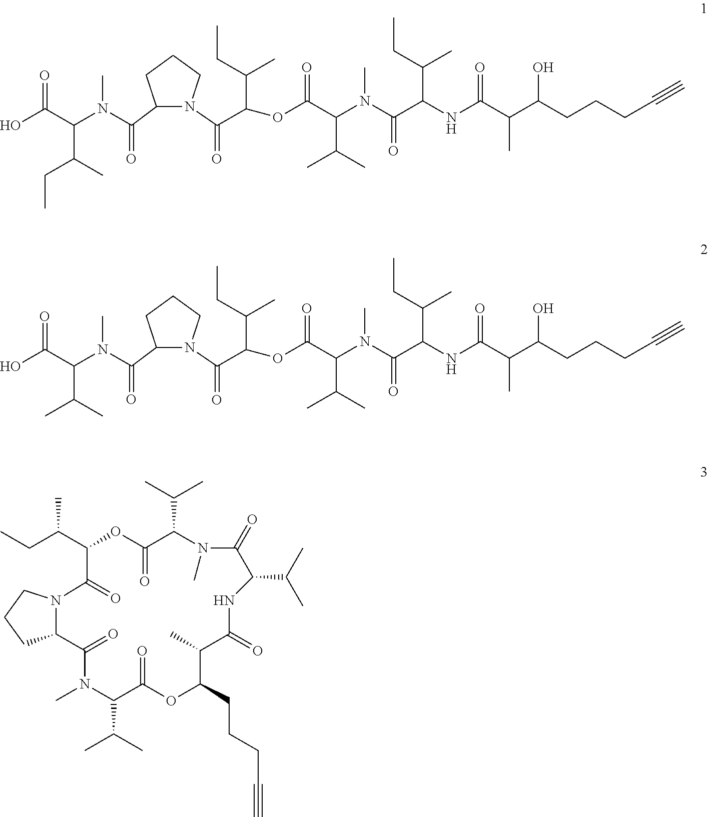

In vitro testing revealed that the veraguamides isolated by the inventors demonstrated disappointing activity in TNBC cells, suggesting that the veraguamides are better suited for development as a non-toxic therapeutic and not for TNBC or other cancers. Advantageously, testing revealed that the veraguamides had affinity for the sigma 2/TMEM97 receptor and in vitro testing in HEK-293 normal cells showed that the isolated compounds had no overall toxicity or effect on the locomotive behavior of the mice. Thus, linear veraguamides and their derivatives may modulate sigma 2/TMEM97 activity and have therapeutic effects in neurodegeneration and pain.

Veraguamides A-G are characterized by the presence of a proline residue, multiple N-methylated amino acids, an α-hydroxy acid, and a C₈-polyketide-derived β-hydroxy acid moiety (“tail moiety”) with a characteristic terminus as either an alkynyl bromide, alkyne, or vinyl group. As hexadepsipeptides, the residues of Veraguamides A-G are not all amide linkages. Instead, at least some of the linkages between residues are ester linkages. For example, veraguamide (C) includes two ester linkages. Broadly speaking, the inventors prepared veraguamide compounds, wherein an ester linkage of the compound backbone was replaced with an amide linkage.

The compounds of the present disclosure include a veraguamide compound or pharmaceutically acceptable salt thereof, wherein the compound is derived from a veraguamide, wherein an ester linkage is replaced with an amide linkage. The compounds may be cyclic or linear. The compounds may include the same proteinogenic amino acids as are present in a veraguamide natural product, except that an ester bond is replaced with an amide bond. In the case of the linear compounds, the compounds may include the same proteinogenic amino acids as are present in the veraguamide natural product, except that an ester bond is replaced with an amide bond and the naturally occurring amino acids may be in any order from the N-terminus and the C-terminus. As used herein, “proteinogenic” refers the 22 standard amino acids encoded by the genetic code in proteins.

The veraguamide compounds may include 5 amino acid residues with a C-terminus and an N-terminus and a tail moiety (as described in more detail within). The tail moiety may be present at the C-terminus or the N-terminus. In a preferred aspect, the tail moiety is present at the C-terminus. The 5 amino acid residue portion of the veraguamide compounds may be represented by the formula -AA₁-AA₂-AA₃-AA₄-AA₅-, wherein one or more of AA₁ to AA₅ is a proline and the remainder of the amino acid residues that are not proline are proteinogenic amino acids, wherein the N of the amide linkages is optionally substituted (e.g., methylated).

In some aspects, the compound includes 4 to 6 amino acid residues comprising a proline and neutral proteinogenic amino acids (e.g., valine, isoleucine). In some aspects, the compound includes 4 to 6 amino acid residues comprising a proline and neutral amino acids wherein the amino acids include a combination of L-amino acids and D-amino acids. In some aspects, the compound includes 4 to 6 amino acid residues comprising a proline and proteinogenic amino acids. In some aspects, the compound includes 4 to 6 amino acid residues comprising a proline and proteinogenic amino acids, wherein the amino acids include a combination of L-amino acids and D-amino acids.

In some aspects, one or more proteinogenic amino acids is replaced with an unnatural amino acid (i.e., “UAA”). Unnatural amino acids are described in Sharma et al. (Unnatural Amino Acids: Strategies, Designs, and Applications in Medicinal Chemistry and Drug Discovery, J. Med. Chem. 67 (22), 19932-19965), incorporated herein by reference. The UAAs may include noncoded amino acids known in native proteins and peptides. For example, post-translational modifications generate nonproteinogenic amino acids as secondary metabolites in microbes (bacteria, fungi), algae, plants, and marine sources. Indeed, in the veraguamide family of compounds, the N of the amide backbone linkage is often methylated. The UAA may be synthetic. For example, a valine may be replaced with a norvaline.

The veraguamide natural product includes a C₈-polyketide-derived β-hydroxy acid moiety with an alkynyl bromide, alkyne, or vinyl group, generally referred to herein as the “tail moiety” (e.g., hydroxyl methyl octynoic acetylene, “HMOYA”). In the compounds of the present disclosure, the tail moiety may include the following groups.

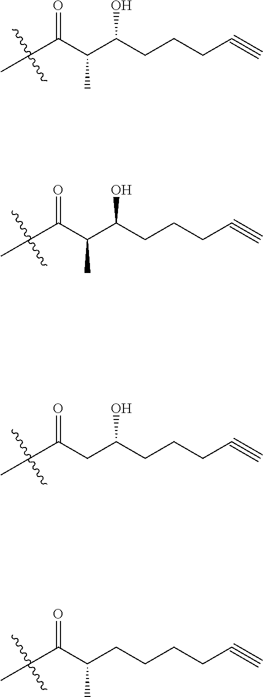 [SMILES: *C(=O)[C@@H](C)[C@H](O)CCCC#C ; *C(=O)[C@H](C)[C@@H](O)CCCC#C ; *C(=O)C[C@H](O)CCCC#C ; *C(=O)[C@@H](C)CCCCC#C]

In some aspects, the tail group may be represented by —C(═O)—C₃-C₁₂ alkyl. The C₃-C₁₂ alkyl group may be optionally substituted, wherein any carbon-carbon single bond of the C₃-C₁₂ alkyl group is optionally replaced by at least one carbon-carbon double or triple bond, and any methylene of the C₃-C₁₂ alkyl group is optionally replaced by at least one O, S, NR, oxo (—C═O), imido (—C═NR), thioxo (—C═S), sulfoxo (S═O), sulfone (S(═O)₂), Se, Ge, or Si. R The “R” group may be hydrogen or a substituent. In some aspects, the tail group may be a C₆-C₁₀ alkyl group wherein one or more carbon-carbon single bonds of the C₆-C₁₀ alkyl group is replaced by a carbon-carbon double or triple bond, and any methylene of the C₆-C₁₀ alkyl group is optionally replaced by at least one O, S, NR, oxo (—C═O), imido (—C═NR), thioxo (—C═S), sulfoxo (S═O), sulfone (S(═O)₂), Se, Ge, or Si. In some aspects, the tail group may be a C₆-C₁₀ alkyl group wherein one or more carbon-carbon single bonds of the C₆-C₁₀ alkyl group is replaced by a carbon-carbon triple bond and the C₆-C₁₀ alkyl group comprises a hydroxyl substituent.

Substituents include:
hydroxyl, deuterated hydroxyl, thiol, deuterated thiol, cyano, halogen, isonitrile (—NC), amino, or nitro; or
a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl (C₂-C₆ heterocycloalkyl), a C₁-C₆ alkyl (C₂-C₆ heterocycloalkenyl), a C₅-C₁₂ aryl, C₂-C₁₂ heteroaryl, a C₁-C₆ alkyl (C₅-C₁₂ aryl), or a C₁-C₆ alkyl (C₂-C₁₂ heteroaryl), optionally substituted with a deuterium, a halogen, or a combination thereof,
wherein any carbon-carbon single bond of the C₁-C₁₂ alkyl and the C₁-C₆ alkyl is optionally replaced by at least one carbon-carbon double or triple bond, and any methylene of the C₁-C₁₂ alkyl and the C₁-C₆ alkyl is optionally replaced by at least one O, S, NR₁₀, oxo (—C═O), imido (—C═NR₁₀), thioxo (—C═S), or Si, wherein R₁₀ is hydrogen or a substituent.

The compounds may have the following structures

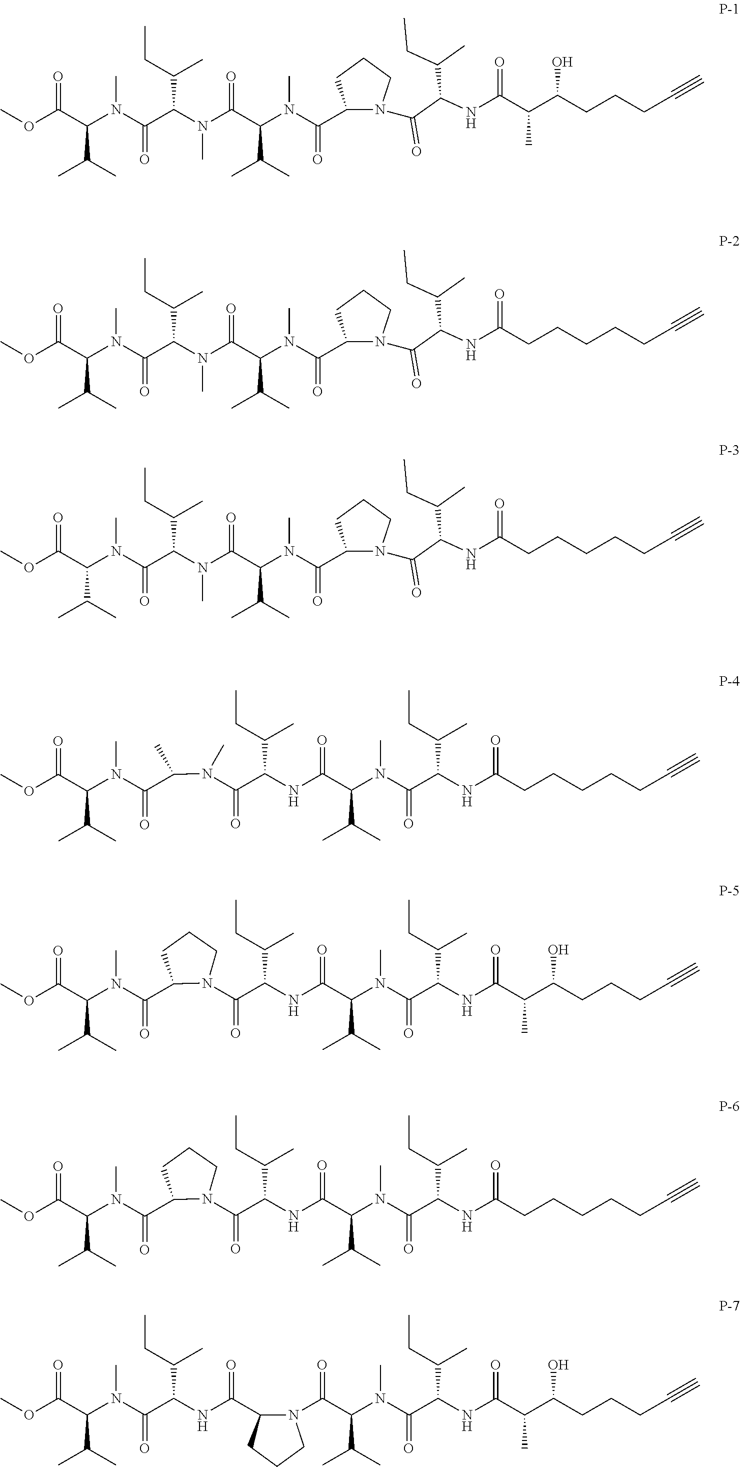

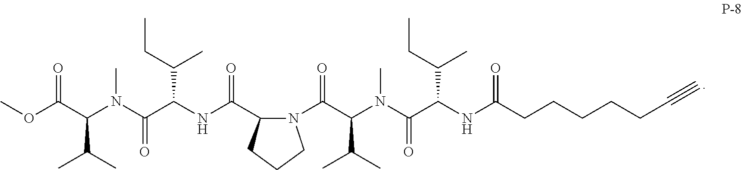 [SMILES: C#CCCCCCC(=O)N[C@H](C(=O)N(C)[C@H](C(=O)N1CCC[C@H]1C(=O)N[C@H](C(=O)N(C)[C@H](C(=O)OC)C(C)C)C(C)CC)C(C)C)C(C)CC]

The presently disclosed compounds also include compounds wherein the peptide backbone is modified. For example, a carbonyl group (C═O) of an amide linkage can be replaced with a thioxo group (C═S) to produce a thioamide linkage. The backbone may be modified in any way was described in Sharma et al. (Unnatural Amino Acids: Strategies, Designs, and Applications in Medicinal Chemistry and Drug Discovery, J. Med. Chem. 67(22), 19932-19965), incorporated herein by reference.

At the C-terminus of the presently disclosed compounds, the carboxylic acid group may be replaced with a bioisostere. For example, a carbonyl group a carboxylic acid group can be replaced with an amide, a hydroxylamide, an ester, a thioester, a hydroxymethyl, or a heterocyclic ring, such as a triazole or a tetrazole.

The presently-disclosed subject matter may include compounds of the formula

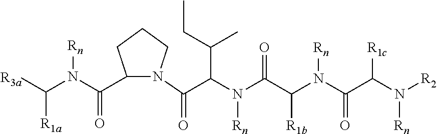

wherein
each occurrence of Rₙ is independently hydrogen, deuterium, or hydrocarbyl;
R₁ₐ, R_{1b}, and Ric are each independently —CH₂(COOH), —(CH₂)₄NH₂, —CH(CH₃)₂, —CH₂—OH, and H;
R₂ is on the following groups; and

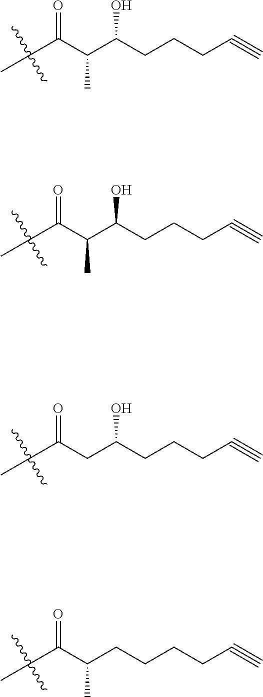 [SMILES: *C(=O)[C@@H](C)[C@H](O)CCCC#C ; *C(=O)[C@H](C)[C@@H](O)CCCC#C ; *C(=O)C[C@H](O)CCCC#C ; *C(=O)[C@@H](C)CCCCC#C]

R₃ is an ester, an amide, a hydroxyl amide, or a tetrazole.

In some aspects, the compounds of the present disclosure include a detectable group. The detectable group may be introduced using click chemistry. One of the most commonly used reactions in click chemistry is the azide-alkyne cycloaddition, in which an azide group (—N₃) reacts with an alkyne group (—C≡C—) to form a triazole ring. Thus, when an alkyne or an azide group is present on the tail moiety, then click chemistry may be used to form the tetrazole group. In some aspects, when the azide is present on the tail moiety, then the fluorescent tag may be introduced by reaction with an alkyne-functionalized fluorescein derivative, a rhodamine dye, or a Cy dye.

The presently-disclosed subject matter further includes a pharmaceutical composition comprising a compound as disclosed herein, a veraguamide natural product (e.g., veraguamide A-G, O, or P), or a combination thereof, and a pharmaceutically acceptable excipient.

The pharmaceutical composition may further comprise an anesthetic adjuvant. The anesthetic adjuvant may include an opioid different from the presently-disclosed compounds, a benzodiazepine, an alpha-2 agonist, an antiemetic, a steroid, or a combination thereof.

The presently-disclosed subject matter further includes a method of treating or preventing pain in a subject comprising administering to the subject a therapeutically effective amount of: a compound as disclosed herein, a veraguamide natural product (e.g., veraguamide A-G, O, or P), or a combination thereof. In some embodiments, the subject has been identified as having chronic pain.

The presently-disclosed subject matter further includes a method of treating a subject having a pain disorder, comprising administering to the subject a therapeutically effective amount of: a compound as disclosed herein, a veraguamide natural product (e.g., veraguamide A-G, O, or P), or a combination thereof. Pain disorders may be associated with nociceptor activity. In some aspects, the compounds disclosed herein and/or the veraguamide natural product may inhibit a nociceptor. By inhibiting nociceptor-like cells, you can reduce or block the transmission of pain signals from peripheral tissues to the central nervous system (CNS). This can lead to significant pain relief in conditions such as chronic pain, neuropathic pain, and inflammatory pain. Neuropathic pain arises from injury or dysfunction of the nervous system, often leading to abnormal activation of nociceptors. Inhibiting nociceptor-like cells or interfering with their activity can help alleviate the intense, burning, or tingling sensations associated with this type of pain. Conventional treatment for neuropathic pain includes certain local anesthetics, anti-inflammatory agents, or neurotoxin treatments (e.g., botulinum toxin). Inflammation can sensitize nociceptors, making them more responsive to stimuli. Inhibiting nociceptor-like cells can reduce the sensitization of pain pathways, helping to manage conditions such as arthritis or inflammatory bowel disease. Therapeutic agents for the treatment of inflammatory pain include nonsteroidal anti-inflammatory drugs (NSAIDs), corticosteroids, or calcitonin gene-related peptide (CGRP) inhibitors. Hyperalgesia refers to an increased sensitivity to pain, and allodynia refers to pain caused by stimuli that are not usually painful. Both of these conditions are often due to the sensitization of nociceptors. Inhibiting nociceptor-like cells can prevent the enhancement of pain sensitivity and reduce discomfort in conditions like fibromyalgia, post-surgical pain, or complex regional pain syndrome (CRPS). Gene therapy or cell-based therapies may offer a way to inhibit specific nociceptor-like cells or receptors selectively by targeting the molecular mechanisms involved in nociception (e.g., TRPV1 receptors or Nav1.7 sodium channels). The TRPV1 receptor is a key ion channel found on nociceptors and is involved in the detection of heat and painful stimuli. Blocking this receptor can reduce the perception of pain. There are ongoing efforts to develop TRPV1 antagonists as a potential therapy for pain management. The Nav1.7 sodium channel is important for the firing of nociceptors. Inhibition of this channel has been shown to reduce pain, particularly in inherited pain disorders like paroxysmal extreme pain disorder (PEPD). Nav1.7 blockers are being studied for their potential to treat conditions like chronic pain and neuropathic pain.

Inhibition of nociceptor cells with a compound as disclosed herein, a veraguamide natural product (e.g., veraguamide A-G, O, or P), or a combination thereof can prevent pain transmission, reduce sensitization, and alleviate hyperalgesia or allodynia, leading to significant therapeutic benefits, particularly for individuals with neuropathic pain, inflammatory pain, or other types of chronic pain. The presently disclosed compounds and/or veraguamide natural products may be used in combination with other therapeutic approaches such as receptor antagonists, ion channel blockers, gene therapies, and biologic treatments are being explored to selectively inhibit nociceptors.

The integrated stress response (ISR) has been shown to be active in nociceptors during chronic pain-like states in rodent models. Inhibition of ISR is a potential mechanism of action for the disclosed compounds. The ISR is a cellular response to various stressors, including nutrient deprivation, oxidative stress, and infection. One of the key regulatory mechanisms in the ISR is the phosphorylation of eukaryotic initiation factor 2α (eIF2α). eIF2α phosphorylation inhibits general protein synthesis while selectively promoting the translation of specific stress-related genes. Under normal conditions, eIF2α promotes the initiation of protein translation by forming a complex with eIF2B and GTP, which binds to the ribosome and allows the translation of mRNA into proteins. However, during stress, kinases such as PERK, PKR, GCN2, and IRE1 phosphorylate eIF2α. Phosphorylated eIF2α (eIF2α-P) prevents the exchange of GTP for GDP, leading to a reduction in general protein synthesis. This serves to conserve cellular resources and reduce the production of new proteins that could contribute to further stress or damage. However, it also selectively promotes the translation of stress-related genes, such as those involved in cell survival, apoptosis regulation, and stress adaptation.

Reducing eIF2α phosphorylation holds potential as a therapeutic strategy for managing chronic pain and acute pain, particularly in conditions involving inflammation, neuropathy, and stress-induced pain. Targeting the pathways involved in eIF2α phosphorylation, such as the PERK kinase or GCN2, may help to restore normal cellular function, reduce hyperalgesia (increased pain sensitivity), and promote neuronal survival. This strategy may provide a novel avenue for developing analgesic therapies that address the underlying mechanisms of pain, especially in diseases where current treatments are ineffective or have undesirable side effects. Phosphorylation of eIF2α is typically induced by stress signals such as inflammation, oxidative stress, or tissue injury, which are often present in pain states. Under these conditions, the ISR is activated to conserve energy and protect the cell from damage, but at the same time, it can influence pain perception through several mechanisms. Chronic pain conditions often involve persistent activation of the ISR, leading to prolonged eIF2α phosphorylation. Reducing eIF2α phosphorylation could prevent the excessive activation of pain pathways, and reduce sensitization in pain neurons. By inhibiting eIF2α phosphorylation, it might be possible to reduce the chronic pain that arises from neuropathic conditions or inflammatory pain.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the presently disclosed subject matter(s) belong.

All patents, patent applications, published applications and publications, GenBank sequences, databases, websites and other published materials referred to throughout the entire disclosure herein, unless noted otherwise, are incorporated by reference in their entirety.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the presently disclosed subject matter as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

The terms “a” and “an” do not denote a limitation of quantity, but rather denote the presence of at least one of the referenced item.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or language indicating an example (e.g., “such as”), is intended merely for illustration and does not pose a limitation on the scope of the presently disclosed subject matter unless otherwise claimed. No language in the specification should be construed as indicating any non-claimed element as essential to the practice of the presently disclosed subject matter.

“Pharmaceutically acceptable salts” include veraguamide compounds modified by making acid or base addition salts thereof, and further refers to pharmaceutically acceptable solvates, including hydrates, and co-crystals of such compounds and such salts. Examples of pharmaceutically acceptable salts include, but are not limited to, mineral or organic acid addition salts of basic residues such as amines; alkali or organic addition salts of acidic residues; and the like, and combinations comprising one or more of the foregoing salts. The pharmaceutically acceptable salts include non-toxic salts and the quaternary ammonium salts of the active agent, a veraguamide compound. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxy maleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts; organic amine salts such as triethylamine salt, pyridine salt, picoline salt, ethanolamine salt, triethanolamine salt, dicyclohexylamine salt, N,N′ dibenzylethylenediamine salt, and the like; and amino acid salts such as arginate, asparginate, glutamate, and the like; and combinations comprising one or more of the foregoing salts.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

A “patient” is a human or non-human animal in need of medical treatment. Medical treatment can include treatment of an existing condition, such as a disease or disorder, prophylactic or preventative treatment, or diagnostic treatment. In some embodiments, the patient is a human patient.

A “therapeutically effective amount” of a pharmaceutical combination of this disclosure means an amount effective, when administered to a patient, to provide a therapeutic benefit such as an amelioration of symptoms, e.g., an amount effective to decrease the symptoms of pain.

The term “anesthetic adjuvant” refers to an active agent that enhances or prolongs the analgesic or anesthetic effect of an analgesic or anesthetic agent.

The presently-disclosed subject matter is further illustrated by the following specific but non-limiting examples. The following examples may include compilations of data that are representative of data gathered at various times during the course of development and experimentation related to the presently disclosed subject matter.

### EXAMPLES

### General Experimental Procedures

NMR spectra were recorded with CD₃CN (δ_{C} 118.2, δ_{H} 1.96), MeOD (δ_{C} 49.0, δ_{H} 3.31) and CDCl₃ (δ_{C} 77.2, δ_{H} 7.26) as internal standards on a Bruker 500 megahertz (MHz) spectrometer (Bruker, Billerica, MA, USA) operating at 499.7 MHz for ¹H and 125.7 MHz for ¹³C equipped with a 5 mm PATXI ¹HD/D-¹³C/¹⁵N Z-GRD Probe. High-resolution mass measurements were obtained using an Agilent Technologies QTOF Accurate Mass mass detector (Agilent Technologies, Santa Clara, CA, USA). Additional mass spectra were acquired with a benchtop Advion mass spectrometer. HPLC separation was carried out using a Dionex Ultimate 3000 pump system (Thermo Fisher, Waltham, MA, USA) with UV detection using HPLC-grade solvent. Accelerated chromatographic isolation was carried out on a Biotage Isolera One system with UV detection using HPLC-grade solvent. Column chromatography was performed using Sorbent Technologies silica gel (Sorbent Technologies, Norcross, GA, USA) (230-400 mesh). Solvents were evaporated on a Heidolph rotary evaporator.

### Collection and Microscopy

Green/grey filamentous cyanobacteria were collected by hand while snorkeling at a depth of ~1 m off the coast of Isla Mina (GPS coordinates: N 8 29.717 W 78 59.947) in the Las Perlas Archipelago, Panama. The sample was fully suspended in 800 milliliters (mL) of 50:50 ethanol:seawater mixture to preserve the bacteria until it could be stored in a light-tight lab at −20° C. prior to extraction. A smaller sample was saved in RNAlater (ThermoFisher) for phylogenetic analysis. A voucher specimen was deposited at the Department of Medicinal Chemistry, Graduate School of Pharmacy, Duquesne University.

Samples of DUQ0008 used for microscopy were prepared using a <1 cubic millimeters (mm³) piece from the RNAlater sample. Filaments were spread out onto a slide with distilled H₂O and imaged at 10× on a (Nikon 1AR-HD confocal microscope, Nikon Instruments, Melville, NY, USA) using the FITC (fluorescein isothiocyanate, 487 nanometers (nm)), TRITC (tetramethylrhodamine isothiocyanate, 560 nm), and Cy5 (cyanine5, 637 nm) and/or bright-field channels.

16S rRNA Gene Sequencing and Phylogenetic Analysis

Genomic DNA was extracted from DUQ0008 that was preserved in RNAlater after collection. Lysis buffer (10 millimoles per liter (mM) Tris, 0.1 M EDTA, 0.5% (w/v) SDS, 20 micrograms per milliliter (μg/mL) RNase, pH 8.0) was added to the cyanobacteria mass at 10× the biomass of the cells (i.e., 100 mg cells=1000 μL lysis buffer). This was followed by 100 μL of lysozyme solution (10 mg/mL, Sigma) for 30 min with an incubation at 37° C., and 0.01× the volume of Proteinase K (10 mg/mL, Gene Link) was then added and incubated for 1 hour (h) at 50° C. Following this, the mixture was centrifuged at 13,000 revolutions per minute (rpm) for 3 minutes (min), and the remaining pellet was used with the Wizard Genomic DNA Purification Kit (Promega, Madison, WI, USA). Extracted genomic DNA then underwent polymerase chain reaction (PCR) to amplify the 16S rRNA gene with previously utilized primers (CYA106F and CYA1509R). Successful PCR amplifications (~1370 base pairs (bp)) underwent PCR purification using the Min Elute PCR Purification Kit (Qiagen, Germantown, MD, USA), and purified PCR products were used for TOPO cloning. One Shot E. coli cells along with the pCR® 4-TOPO vector with kanamycin resistance from the TOPO TA Cloning® Kit (Invitrogen, Waltham, MA, USA) were used, and cells were plated overnight at 37° C. on LB-kanamycin plates. Successful transformants were selected and used in colony PCR with the M13F and M13R primers to confirm the presence of the 16S rRNA sequence in the TOPO vector (~1535 bp). The purified PCR product for correct samples was sent to Beckman Coulter (now GENEWIZ) to undergo forward and reverse Sanger sequencing with the M13F and M13R primers. The sequence is available in the GenBank database under the accession number MH345835. The sequence for DUQ0008, along with 16S rRNA gene sequences that were obtained with sequence data available at the National Center for Biotechnology (NCBI) webpage (http://www.ncbi.nlm.nih.gov, accessed on 25 Mar. 2018), were then used for phylogenetic analysis. The DUQ0008 gene sequence was first aligned in an unbiased fashion using the NCBI Standard Nucleotide BLAST to determine the closest alignment. Five of the top six alignment hits were from the newly characterized Okeania genus. The DUQ0008 was aligned with the top NCBI BLAST Okeania hit and all Okeania type gene sequences from a recent phylogenetic analysis of natural product-producing cyanobacteria. Sequences were aligned using the Muscle algorithm, and phylogeny reconstruction was carried out using the maximum likelihood method with 600 bootstrap replicates via the MEGA (version 5.1) program.

### Compound Extraction and Isolation

The cyanobacterial biomass (75 grams (g), dry weight) was extracted exhaustively with 2:1 CH₂Cl₂-MeOH to afford 3.4 g of crude extract. This crude extract was fractionated over normal-phase silica gel with a stepwise gradient solvent system of increasing polarity, starting from 100% hexanes to 100% MeOH, to yield nine fractions (A-I). The fraction eluting with 100% MeOH (fraction I, 150 mg) was chromatographed over a Hypersep C18 (1000 milligram (mg), 8 mL) cartridge, and the column was eluted with 30 mL of 50% and 75% MeOH-water mixture, then 100% MeOH to yield sub-fractions I1-3, corresponding to 50.7, 73.1, and 13.9 mg, respectively. A portion of the fraction eluted with 50% methanol in water (sub-fraction I1, 30.3 mg) was subjected to accelerated reversed-phase (Biotage, Snap KP-C18-HS, 12 g cartridge) chromatography using a linear gradient of acetonitrile-water (50% acetonitrile for 9 column volumes followed by 50-100% acetonitrile for 8 column volumes, and then 100% acetonitrile for 9 column volumes). These 22 chromatographic fractions were combined into five subfractions (I1A-E) based upon peaks observed in the chromatogram generated from UV detection at 210 and 254 nanomoles per liter (nM). Sub-fraction IID (9.4 mg) was subjected to reversed-phase HPLC (Phenomenex, Synergi-fusion 4μ, 150×10 mm) using a linear gradient of acetonitrile-H₂O acetonitrile-0.1% TFA (in water) gradient (55% acetonitrile for 22 min, then 55-100% acetonitrile in 5 min, and then 100% acetonitrile for 5 min) to yield veraguamide O (1) (2.3 mg, 14.24 min) and veraguamide P (2) (0.3 mg, 11.17 min). Sub-fraction ILE, which comprised a mixture of 1 and 2 (3.9 mg), as confirmed by 1H NMR and mass spectral analysis, was chromatographed under similar conditions to afford additional veraguamide O (1) (1.5 mg, 14.33 min) and veraguamide P (2) (0.5 mg, 11.25 min).

Fraction G (564.8 mg) from the original A-I fractionation was subjected to silica gel column chromatography and yielded seven fractions, G1-7. Sub-fraction G1 (52.3 mg) was further chromatographed over reversed-phase HPLC (Phenomenex, Torrance, CA, USA, Synergi-fusion 4u, 150×10 mm; 83% MeOH:H2O for 20 min, followed by 100% MeOH for 10 min; 1.5 mL/min) to afford a sub-fraction (12.0 mg), which was collected between 25.0 and 30.0 min. This sub-fraction was subjected to accelerated reversed-phase (Biotage, Salem, NH, USA, Snap KP-C18-HS, 12 g cartridge) chromatography using a linear gradient of methanol-water (50% MeOH in 2.5 column volumes then 50-100% in 16 column volumes; 30 mL/min). The fraction collected at 10 column volumes afforded veraguamide C (3) (1.7 mg) after concentration in vacuo.

Veraguamide O (1): colorless oil; ¹H-, ¹³C- and 2D-NMR see Table 1; HRESIMS m/z 773.4361 [M+K] (calcd, for C₃₉H₆₆N₄O₉K, 773.4466), m/z 757.4626 [M+Na] (calcd. For C₃₉H₆₆N₄O₉Na, 757.4727) and m/z 735.4804 [M+H] (calcd, for C₃₉H₆₇N₄O₉, 735.4908).

Veraguamide P (2): colorless oil; ¹H-, ¹³C- and 2D-NMR see Table 1; HRESIMS m/z 743.4626 [M+Na] (calcd, for C₃₈H₆₄N₄O₉Na, 743.4571).

Veraguamide C (3): colorless oil; ¹H-NMR was comparable to previously reported literature values. ESIMS m/z 733.98 calcd, for C₃₇H₉₅N₄O₉Na₂ [M+2Na−H].

### Methanolysis/Partial Hydrolysis of Veraguamide O and Veraguamide P

A sample of Veraguamide O (1) (0.5 mg) was treated with 5% NaOH (v/v) in methanol at room temperature for 24 h. The reaction mixture was concentrated to dryness, and the residue was partitioned between H₂O and EtOAc. The organic layer was collected and dried, and the identity of the crude methanolysis products was confirmed by ESI-MS. A similar procedure was repeated for Veraguamide P (2).

### Cell Culture

The human triple-negative breast cancer cell line MDA-MB-231 was obtained from ATCC and was maintained in DMEM:F-12 (1:1) (Life Technologies, Carlsbad, CA, USA), 10% Fetal Bovine Serum (Atlanta Biologicals, Atlanta, GA, USA), and 1% Penicillin/Streptomycin (Sigma, St. Louis, MO, USA). The estrogen-positive MCF-7 cell line was also obtained from ATCC and was maintained in 1640 RPMI media (Life technologies), 10% FBS, and 1% Penicillin/Streptomycin. HEK-293 cells were also obtained from ATCC and maintained in Dulbecco's Modification of Eagle's Medium (DMEM), 10% Fetal Bovine Serum (FBS), and 1% Penicillin/Streptomycin (Pen/Strep).

### Cytotoxicity Assays

Two rounds of toxicity experimentation were completed. First, MDA-MB-231 and MCF-7 cells were plated at a density of 5×10³ cells per well in a 96-well plate. Cells were allowed to attach overnight. The cells were treated with depsipeptides or vehicle for 72 h with 5% FBS stimulation. DMSO was used as a vehicle control. After treatment, 10 microliters (μL) MTT (Sigma) was added to each well (0.5 mg/mL final concentration), and the plates were incubated for 3 h (5% CO₂ and 37° C.). The medium was removed and the MTT-formazan crystals were dissolved with 100 μL DMSO per well. The absorbance was measured at 570 nm with a VICTOR3 1420 multilabel counter (Perkin Elmer, Springfield, IL, USA). Three wells were analyzed for each condition, and wells containing medium-MTT only (no cells) and medium-MTT (DMSO+5% FBS) were used as controls. Results were normalized to the DMSO+5% FBS group.

Second, HEK-293 cells were plated at a density of 3×10⁴ cells per well in a 96-well plate. Cells were allowed to attach overnight. The cells were treated with veraguamides C and P (or vehicle 0.5% DMSO). Veraguamides were suspended in DMEM to achieve final concentrations ranging from 0.01 μM to 10 μM in 0.5% DMSO (final concentration in well). Eleven replicate wells were used for each concentration of each treatment. Cells were treated for 24 h. After 24 h of exposure to the test compounds, cell viability was assessed using the CellTiter-Blue® assay. Briefly, 20 μL of CellTiter-Blue® reagent was added directly to each well containing 100 μL of media. The plates were then incubated for an additional 4 h at 37° C. to allow for sufficient metabolic conversion. The fluorescence was measured using a fluorescence microplate reader set to an excitation wavelength of 560 nm and an emission wavelength of 590 nm. The percentage of viable cells was calculated by comparing the fluorescence readings of the treated wells (11 per concentration) with those of the control DMSO wells.

### Psychoactive Drug Screening Program (PDSP) Receptor Screening

All binding data for fractions and compounds were obtained and generously provided by the National Institute of Mental Health's Psychoactive Drug Screening Program (NIMH PDSP) utilizing a radioligand competition-binding assay. In brief, fractions that passed a primary binding criterion of greater than 50% inhibition at 10 M were tagged for secondary radioligand binding assay to determine the apparent or real IC₅₀. Data reported here detail the affinity of fractions and compounds to the sigma receptors.

### In Vivo Testing

All animal procedures were reviewed and carried out in accordance with the National Institutes of Health Guide for the Care and Use of Animals and the Institutional Animal Care and Use Committee at Duquesne University. Experiments were performed on male C57Bl/6J mice that were 8-10 weeks old.

Surgical Procedures. Animals were anesthetized with 3% isoflurane/0.6% O₂ during all surgical procedures. Intracerebroventricular (ICV) cannulation surgeries were performed. Briefly, mice were placed in a stereotaxic frame and an 8.00 mm steel cannula was placed into the right lateral ventricle at the following anatomical coordinates: 0.5 mm anterior to bregma, 1.0 mm lateral to midline, and 2.0 mm ventral to the skull. A dental cement skullcap secured with two bone screws was used to hold the cannula in place. Mice recovered on heating pads and were given one week of recovery prior to the beginning of behavioral testing. Following behavioral testing, cannula placement was verified with necropsy.

Open Field. Open field was completed. Briefly, mice were habituated to a dimly lit room for 1 h with 60 dB white noise. Veraguamide C (3) was dissolved in a mixture of 50% DMSO and 50% artificial cerebrospinal fluid (aCSF) containing the following (in mM): 125 NaCl, 2.5 KCl, 1.25 NaH₂PO₄, 25 NaHCO₃, 2.0 CaCl₂, 1.0 MgCl₂, and 25 d-glucose, bubbled with 95% O₂/5% CO₂ for 20 min prior to use. Microinjections were performed using a 32-gauge injector that extended 0.5 mm beyond the tip of the ICV cannula. The injector was attached to flexible tubing and a 1.0 μL syringe (Hamilton, Reno, NV, USA) that was used to deliver a total volume of 0.5 μL over a 2 min period. The injector was kept in place for an additional 1 min to allow for complete compound infusion. Veraguamide C (3) was administered at a 20 μg dose, and 50%/50% aCSF/DMSO (0.5 μL) was used as a vehicle control. Mice were treated with veraguamide C (3) or vehicle, 5 min before testing in the open field box (40.6×40.6×30 cm). Behavior was videorecorded and scored using ANY-Maze software (Stoelting Co., Wood Dale, IL, USA, Version 4.98). Total distance travelled (meters, m) was measured. Injection and analysis were completed while blinded to treatment.

### Example 1. Compound Isolation and Identification from Cyanobacterial Collection

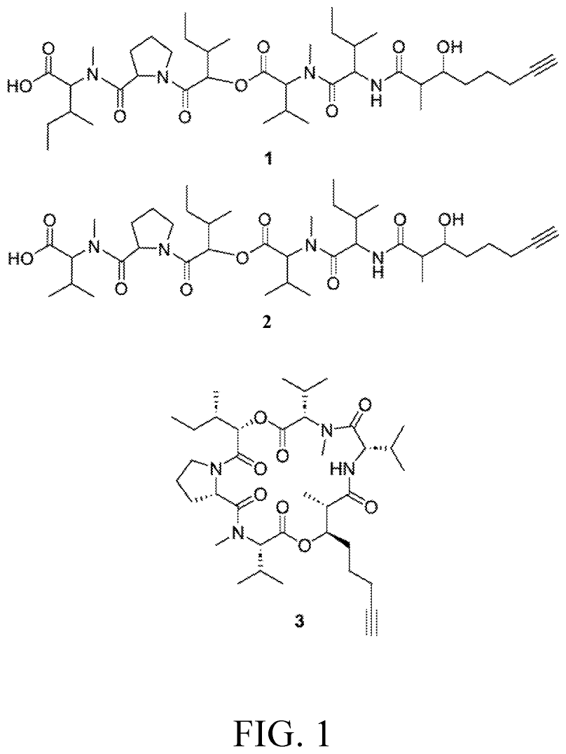

A cyanobacterial mat was collected off the coast of Isla Mina in the Las Perlas Archipelago, Panama (GPS coordinates: N 8 29.717 W 78 59.947), and given the extraction ID of DUQ0008. This collection was extracted with CH₂Cl₂-MeOH (2:1) to afford 3.3 g of crude extract. This crude extract was fractionated by silica gel chromatography to yield nine fractions labeled A (100% hexanes) through I (100% methanol). Fraction I, eluted with 100% methanol, was further purified using accelerated chromatography and reversed-phase HPLC to afford veraguamide O (1) (3.8 mg) and veraguamide P (2) (0.8 mg) (FIG. 1); Fraction G, eluted with 10% hexanes/90% methanol, was further purified using accelerated chromatography and reverse-phase HPLC to afford veraguamide C (3) (1.7 mg) (FIG. 1). All were recovered as colorless oils.

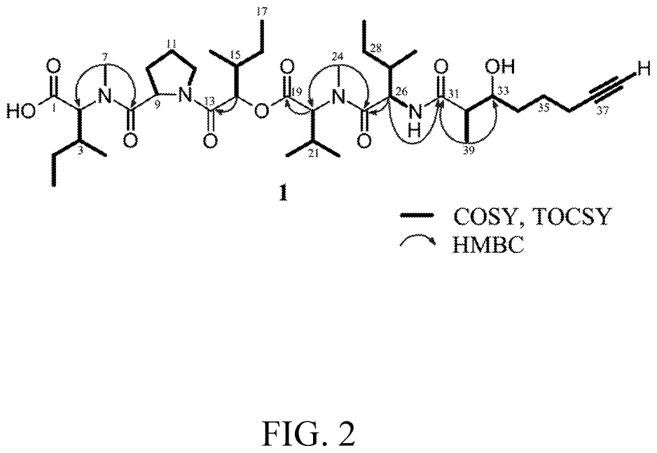

HRESIMS of (1) in positive mode provided molecular ion peaks at m/z 735.4804 [M+H], and 757.4626 [M+Na], suggesting a molecular formula of C₃₉H₆₆N₄O₉, corresponding to nine degrees of unsaturation. The structure of veraguamide O (1) was established by analysis and correlations of peaks in the NMR spectra, which revealed that it was a depsipeptide (Table 1), with α-protons (δ_{H} 4.67-4.87), an exchangeable amide (NH) proton (δ_{H} 6.82), two N-methyl groups (δ_{H} 3.11 and 3.06), and deshielded signals in both the ¹H and ¹³C NMR spectra typical of oxymethines (8H/C 4.95/76.5 and 3.60/71.7), one of which was adjacent to an ester linkage. These observations were further supported by ¹³C NMR resonances derived from HSQC and HMBC experiments, assignable to six ester, amide, and carboxylic acid carbonyls (δ_{C} 175.6, 173.1, 170.3, and 170.1). The carbonyl groups together with the terminal alkyne (8 84.4, 68.7, δ_{H} 2.16) of the 3-hydroxy-2-methyloctynoic acid (Hmoya) residue accounted for eight of the nine degrees of unsaturation and suggested that one possesses an additional ring. Further analysis of the COSY, TOCSY, HSQC, and HMBC data for veraguamide O (1) in CD₃CN (Table 1 and FIG. 2) revealed the presence of four proteinogenic amino acid residues (N-Me-Ile, Pro, N-Me-Val and Ile) and the hydroxy acid, 2-hydroxy-3-methylpentanoic acid (hmpa). Given that the proline residue accounted for the final degree of unsaturation, and that there are two terminal groups (N-Me-Ile and Hmoya), it was clear that the compound is linear. The fragments N-Me-Ile-Pro-Hmpa-N-Me-Val-Ile-Hmoya were readily constructed with the help of TOCSY and HMBC data.

TABLE 1
NMR Spectroscopic Data for Veraguamides O and P in CD₃CN

| | | Veraguamide O | | | Veraguamide P | | |
|---|---|---|---|---|---|---|---|
| Residue | Pos. | δ_{C}, Type | δ_{H} (J in Hz) | HMBC ^{a} | δ_{C} | δ_{H} (J in Hz) | HMBC ^{a} |
| N-Me-Ile ^{d}/ | 1. | 170.3, C | | | 170.3 | | |
| N-Me-Val ^{e}-1 | 2. | 61.5, CH | 4.67, d (10.7) | 7 | 61.5 | 4.70 | |
| | 3. | 32.6, CH | 2.01, m | | 27.1 | 2.23 | |
| | 4. | 24.6, CH₂ | 1.15 | | — | — | |
| | 5. | 15.1, CH₃ | 0.97 | 2, 3, 4 | 19.0 | 0.90 | 2, 3, 6 |
| | 6. | 9.9, CH₃ | 0.87 | 3, 4 | 18.1 | 0.80 | 2, 3, 5 |
| | 7. | 31.7, CH₃, | 3.06, s | 2, 8 | 31.6 | 3.06, s | 8 |
| Pro | 8. | 173.1, C | | | 172.9 | | |
| | 9. | 56.8, CH | 4.79, q (8.1, 5.2) | | 56.7 | 4.80 | |
| | 10. | 28.2, CH₂ | 1.76, m | | 28.2 | 1.79, m | |
| | 11. | 24.5, CH₂ | 2.23, 2.09 | | 24.5 | 2.22, 2.08 | |
| | 12. | 47.2, CH₂ | 3.83, m | | 47.0 | 3.82, m | |
| | | | 3.60 ^{c} | | | 3.61 ^{c} | |
| Hmpa | 13. | 170.3, C | | | 170.3 | | |
| | 14. | 76.5, CH | 4.95 | 13, 15, 16, 18 | 76.2 | 4.90 | |
| | 15. | 36.2, CH | 1.93 ^{b} | | 36.0 | 1.93 ^{b} | |
| | 16. | 24.1, CH₂ | 1.67, s | | 23.8 | 1.67 | |
| | 17. | 10.4, CH₃ | 0.89 | 15, 16 | 10.3 | 0.89 | 15, 16 |
| | 18. | 14.0, CH₃ | 1.00 | 14, 15, 16 | 13.8 | 0.99 | 14, 15, 16 |
| N-Me-Val-2 | 19. | 170.1, C | | | 170.3 | | |
| | 20. | 61.7, CH | 4.87, d (10.7) | 19, 21, 23, 24 | 61.6 | 4.86, d (10.6) | 19, 21 |
| | 21. | 27.1, CH | 2.23 | | 27.1 | 2.26 | |
| | 22. | 19.1, CH₃ | 0.99 | 20, 21, 23 | 19.0 | 0.99 | 20, 21, 23 |
| | 23. | 17.9, CH₃ | 0.79, d (6.7) | 20, 21, 22 | 18.1 | 0.79 | 20, 21, 23 |
| | 24. | 31.3, CH₃ | 3.11, s | 20, 25 | 31.4 | 3.12 | 20 |
| Ile | 25. | 173.1, C | | | 173.0 | | |
| | 26. | 52.9, CH | 4.70 ^{c} | 25, 27, 31 | 52.8 | 4.70, m | |
| | 27. | 36.9, CH | 1.81, m | | 36.8 | 1.83, m | |
| | 28. | 24.5, CH₂ | 1.57, 1.15 | | 24.5 | 1.59, 1.17 | |
| | 29. | 10.4, CH₃ | 0.90 | | 10.3 | 0.90 | 27, 28 |
| | 30. | 14.9, CH₃ | 0.90 | 26 | 14.9 | 0.91 | 26, 27, 28 |
| Hmoya | 31. | 175.6, C | | | 175.5 | | |
| | 32. | 45.4, CH | 2.35, m | 31, 33 | 45.1 | 2.34, m | 31, 39 |
| | 33. | 71.7, CH | 3.60, m | | 71.6 | 3.61, m | |
| | 34. | 32.8, CH₂ | 1.42 | | 32.7 | 1.41 | |
| | 35. | 24.6, CH₂ | 1.67, 1.48 | | 24.9 | 1.67, 1.47 | |
| | 36. | 17.9, CH₂ | 2.19 | 34, 35, 37 | 17.7 | 2.18 | 34, 35, 37, 38 |
| | 37. | 84.4, C | | | 84.5 | | |
| | 38. | 68.7, CH | 2.16 ^{b} | 37 | 68.7 | 2.16 ^{b} | |
| | 39. | 12.1, CH₃ | 1.09, d (6.6) | 31, 32, 33 | 11.9 | 1.10, d (6.6) | 31, 32, 33 |
| | | NH | 6.82, d (9.0) | | | 6.81, d (8.6) | |
| | | OH | 3.43 | 34 | | | |

^{a} Carbon correlating to proton shift,
^{b} signal obscured,
^{c} signal partially obscured,
^{d} pertaining to 1,
^{e} pertaining to 2

The continuous sequence of the residues was determined by long-range HMBC connectivities (FIG. 2) and mass spectral analysis of the partial hydrolysis product of veraguamide O (1). ESI-MS analysis of the partial hydrolysis product of (1) (Scheme 1) provided evidence for molecular ions at m/z 379.3 [M+Na], 355.3 [M−H], 419.3 [M+Na], and 395.3 [M−H]. These corresponded to fragments with molecular formulas C₁₈H₃₂N₂O₅ (Scheme 1, Compound 5) and C₂₁H₃₆N₂O₅ (Scheme 1, Compound 7), respectively. The connectivity between the N-Me-Ile and Pro residues was established by HMBC correlations between the hydrogens of the N-Me (δ 3.06) and the carbonyl carbon at C-8 (δ 173.1), indicating an amide linkage. These correlations, together with information derived from the mass of fragment 5 (Scheme 1, compound 5), suggest that the hmpa residue was linked to the proline containing dipeptide fragment. The connection between hmpa and N-Me-Val was inferred from ester hydrolysis of veraguamide O (Scheme 1, compound 1). Next, HMBC cross-peaks were observed between the N-Me hydrogens of N-Me-Val (δ 3.11) and C-25 (δ 173.1) of Ile. Lastly, the α-hydrogen at H-26 (δ 4.70) linked the latter residue to the terminal Hmoya unit through HMBC connectivity to the carbonyl carbon at δ 175.6 (C-31).

Compound 2 (FIG. 1), also a depsipeptide, as suggested by its ¹H NMR spectrum, displayed molecular ion peaks that indicated the molecular formula C₃₈H₆₄N₄O₉, which also corresponded to nine degrees of unsaturation. The six residues, which accounted for all nine degrees of unsaturation, were readily identifiable from characteristic ¹H and ¹³C NMR resonances and from TOCSY and HMBC correlations (Table 1). These data, together with the molecular formula, provided evidence for a 3-hydroxy-2-methyloctynoic acid (Hmoya), a 2-hydroxy-3-methylpentanoic acid (hmpa), and four α-amino acid residues (two N-MeVal, Pro and Ile). Combining the molecular formula and NMR data for Veraguamide P suggested that it was related to Veraguamide O, differing by 14 mass units, and this provides the justification for its linear structure. The difference of 14 mass units could be explained by the loss of a CH₂ unit. The loss of the CH₂ unit likely occurred at the pendant alkyl groups of proteogenic or hydroxy acid side chains. This is supported by (1) the chemical shifts for two N-Me (δH/δC 3.11/31.3 and 3.06/31.7), COSY correlations of the sequence H-39-H-32-H-33-H-34-H-35-H-36-H-38, which accounted for all three methylene groups of the terminal Hmoya residue, and (2) the fact that there are no additional chemical shifts for an amide proton (NH). The position of the CH₂ loss was confirmed by mass analysis of the partial hydrolysis product of veraguamide P (Scheme 1, compound 2), which, like veraguamide O, provided evidence for fragment 7 (Scheme 1, compound 7) (m/z 419.3 [M+Na] and 395.3 [M−H]). This suggested that either the hmpa or N-Me-Ile was replaced by 2-hydroxy-3-methylbutanoic acid (hmba) or N-Me-Val-1 residue, respectively. A TOCSY experiment readily linked the α-proton (8 4.90) signal to a spin network belonging to an hmpa residue. HMBC cross-peaks from the hydrogens of the gem-dimethyl group to each other and to the α-carbon at C-2 (δ 61.5) established that the N-Me-Ile residue is replaced by the N-Me-Val-1 in (2) (Table 1).

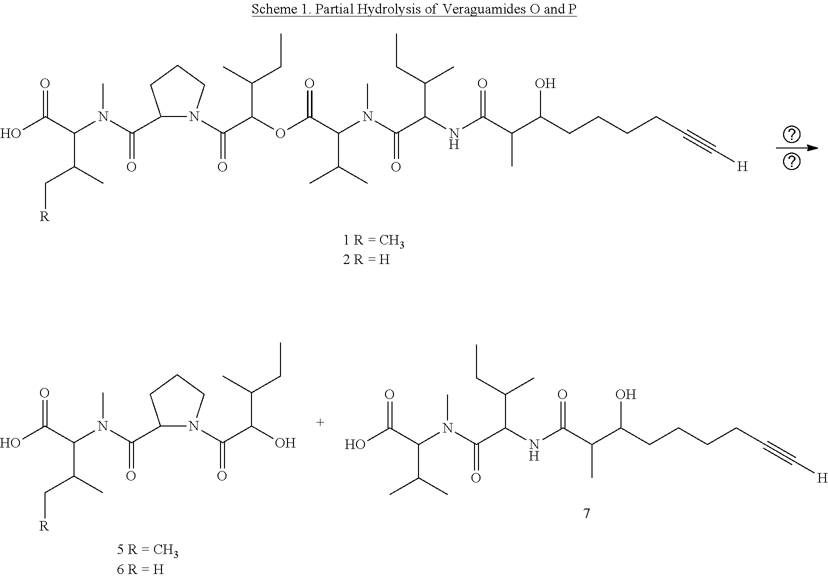

Veraguamide C (1) (FIG. 1) was isolated as a colorless oil after purification by HPLC and accelerated chromatography from the G fraction of the DUQ0008 extract. The veraguamide C was identified by comparison to literature values for samples of veraguamide originally isolated from Oscillatoria margaritifera collected in Panama. The six residues identified in veraguamide O and P and in particular the Hmoya unit, are consistent with linear and cyclo-hexadepsipetides of the veraguamide series of compounds. These observations, together with the recovery of veraguamide C from fraction G (a less polar fraction to I), suggested that the isolated compounds (1) and (2) were related to veraguamides. Comparative analysis of the residue sequence of these linear depsipeptides with the veraguamides and other members of the kulolide family indicated that the compounds are new. The isolated compounds (1) and (2) were assigned the trivial names veraguamide O and veraguamide P, respectively.

The stereoconfiguration was not determined due to limited material; however, a comparison of the ¹³C NMR data to those of veraguamides A and C and the linear depsipeptide veraguamide L, revealed only L-amino acid residues, as well as L-hmpa and 2S, 3R, -Br-Hmoya after Marfey's reagent derivatization and formation of esters, respectively. Based on the analysis and literature values, the veraguamides are expected to contain the same stereo-configuration as previously reported analogs. However, only the planar structure is reported with high confidence.

### Morphological and Phylogenetic Analysis of DUQ0008

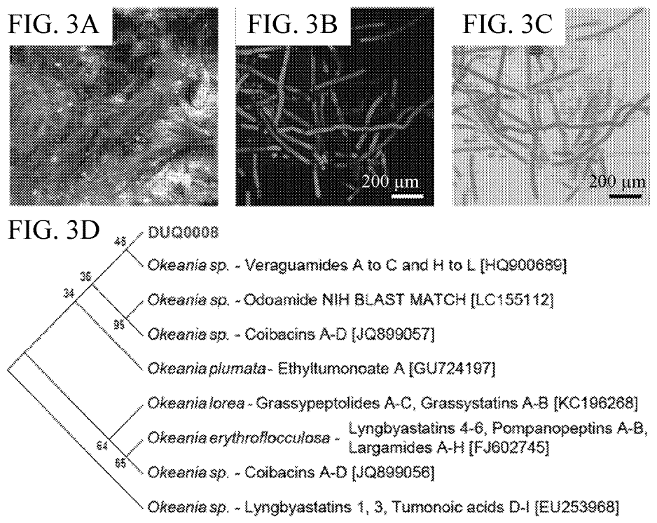

The DUQ0008 collection was a dark green/grey marine cyanobacterial mat collected in ~1 m of water off the coast of Isla Mina in the Las Perlas Archipelago, Panama (FIG. 3A). Confocal (FIG. 3B) and transmitted light (FIG. 3C) microscopy were performed in order to visualize the microscopic structure of the organism. Based on the morphology of the specimen, this sample was field-identified as belonging to the genera of Oscillatoria or Moorea. In order to genetically determine the species of marine cyanobacteria, sequencing of the 16S rRNA gene was performed. The DUQ0008 16S rRNA sequence was first compared in an unbiased fashion using the NCBI Standard Nucleotide BLAST. The top hit identified with this search (99% alignment) was a species in the Okeania genus that produces a cytotoxic cyclohexadepsipeptide, odoamide. A phylogenetic tree was built using a collection of sequences, from Engene et al. Appl. Environ. Microbiol. 2013, 79, 1882-1888, that highlights the relatedness of natural product producing marine cyanobacterial strains (FIG. 3D). The closest related species to DUQ0008 were based on 16S rRNA sequencing with comparison to natural product producing cyanobacteria from the Okeania genus. Compounds produced by species are indicated if available, accession numbers are provided in brackets and numbers next to the tree show the percent of trees showing alignment in 600 replicates. In this tree, the DUQ0008 groups most closely aligned with an Okeania species that produces veraguamides A to C and H to L (FIG. 3D). This relationship is consistent with the compound analysis above, since DUQ0008 also produced veraguamide C. One of the previously collected veraguamide producers and the collection of DUQ0008 both came from the Pacific side of Panama, indicating a possible shared common ancestor of these species.

### Analysis of Veraguamides O, P, and C in Breast Cancer and HEK-293 Cells

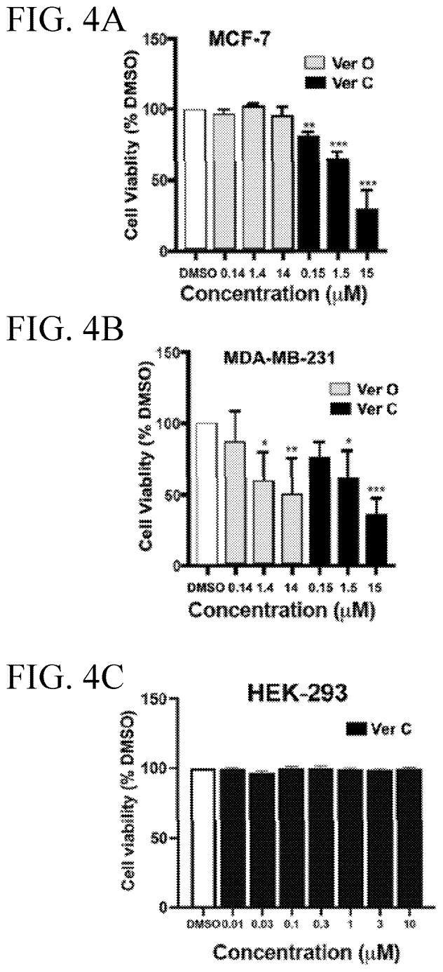

Next, the isolated veraguamides were tested to determine if they would produce cytotoxic effects in cell lines. Veraguamides O (1), P (2), and C (3) were assessed for their effects against human MCF-7 (estrogen positive), MDA-MB-231 (TNBC) breast cancer, and/or HEK-293 (human embryonic kidney) cell lines. The linear depsipeptide veraguamide O (1) displayed selective inhibitory effects against TNBC (MDA-MB-231) cells compared to estrogen-positive (MCF-7) breast cancer cells using a MTT assay (FIG. 4A and FIG. 4B). Veraguamide O (1) decreased viability in MDA-MB-231 cells in a concentration-dependent manner from 0.14 μM (86% viability) to 14 μM (43% viability), and the IC₅₀ of (1) was determined to be 13±4 μM. Functional results from the MTT assay were confirmed microscopically, where veraguamide O (1) appeared to induce cell death in TNBC cells (MDA-MB-231) at 10 μM. The cyclic depsispeptide veraguamide C (3) exhibited similar results in both cell lines, causing cytotoxicity of 53% in TNBC cells and 65% in MCF7 cells at 15 μM. Values for veraguamide C (3) against MCF-7 and MDA-MB-231 cell lines were comparable to those reported previously in other cell lines. In HEK-293 cells, veraguamide C (3) showed no toxicity up to 10 μM (FIG. 4C). The data in FIGS. 4A to 4C represent the mean value+/−standard error (SE) of three different experiments, * indicates p<0.05, ** indicates p<0.01, *** indicates p<0.001 versus a control group as determined by one-way ANOVA with the Bonferroni post hoc test.

While these values did not rise to a level to be considered strong cytotoxic leads, it is interesting to note the difference in activity between the cyclic and linear analogs. To our knowledge, maedamide was the only linear depsipeptide that exhibited antitumor cytotoxicity in the cell culture. Given the low potential for developing these as cytotoxic agents, the potential binding to membrane-bound receptors was assessed to determine if the differential activity was due to a modulation of the cells through a specific receptor.

### Receptor Binding Affinity

The parent fractions (fractions G and I), as well as pure natural products (1) and (3), were screened against membrane-bound receptors by the Psychoactive Drug Screening Program (PDSP) (Table 2). The compounds tested only showed strong binding to the simga2/TMEM97 receptor. Veraguamide C (3) was found to bind with an IC₅₀ of 320 nanomoles per liter (nM), while veraguamide O (1) showed an IC₅₀ of 720 nM. This affinity for the sigma 2/TMEM97 receptor is interesting in light of the cancer cell data because this target has been shown to be upregulated and important in activity against MDA-MB-231 cells. This target has also been shown to be involved in activation of the Store Operated Calcium Entry (SOCE) pathway within MDA-MB-231 cells, leading to apoptosis.

TABLE 2
Binding affinity results for fractions and pure compounds against
the Sigma 2 receptor (Transmembrane protein 97 (TMEM97)).

| | Primary: | Secondary: | |
|---|---|---|---|
| | Percent Inhibition of Binding | IC₅₀ | |
| Fraction G | 60% | 1085 | ng/mL |
| Fraction H | 54% | 1874 | ng/mL |
| Fraction I | 68% | 424 | ng/mL |
| Veraguamide C (3) | 65% | 316 | nM |
| Veraguamide O (1) | 87% | 721 | nM |

Fractions and compounds that pass primary binding criterion of greater than 50% inhibition at 10 μM are tagged for secondary radioligand binding assay to determine apparent (fraction) or real (compound) IC₅₀.

### Analysis of Overt Toxicity Due to Veraguamide C (3) in Mice

The cytotoxicity data above showed minimal effects of the veraguamides on non-TNBCs. These data suggest that the veraguamides would generally be safe and not exert overt toxicity in naïve animals. To test this hypothesis, a limited pilot blinded experiment was completed looking at the locomotor behavior of mice following an intracerebroventricular (ICV) injection of 20 μg (0.5 μL volume) of veraguamide C (3) compared to a vehicle. In a 10 min open field trial, there was no effect of veraguamide C (3) on the total distance traveled compared to the vehicle (veraguamide C (3) 35 m±4 sd, n=4 vs. control 31 m±10 sd, n=4; unpaired t-test p>0.05).

These results indicate potential use of veraguamide analogs as non-toxic sigma receptor-targeting agents.

### Synthesis of 3-Hydroxy-2-Methyloctynoic Acid (HMOYA)

HMOYA was prepared as shown in the following synthetic scheme (Scheme 2).

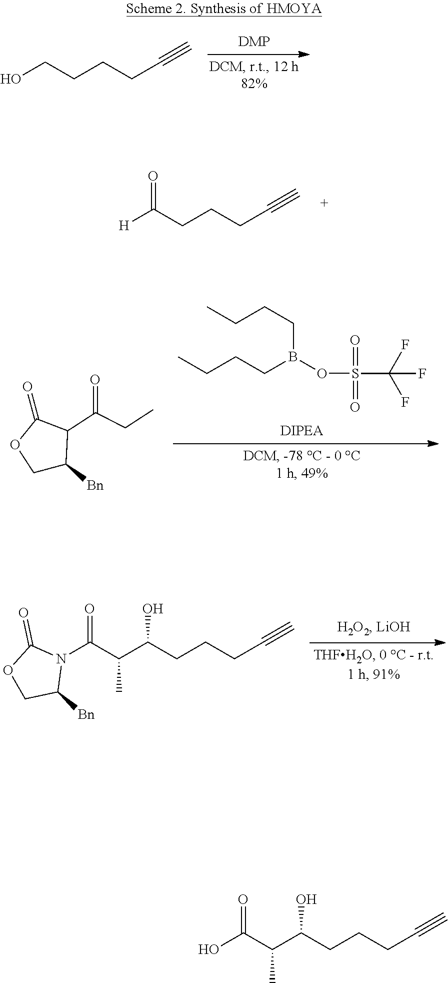

### Synthesis of Peptide Backbone and Veraguamide P Amide

A representative synthesis of the peptide backbone and veraguamide (i.e., veraguamide P (2)) amide is shown in Scheme 3.

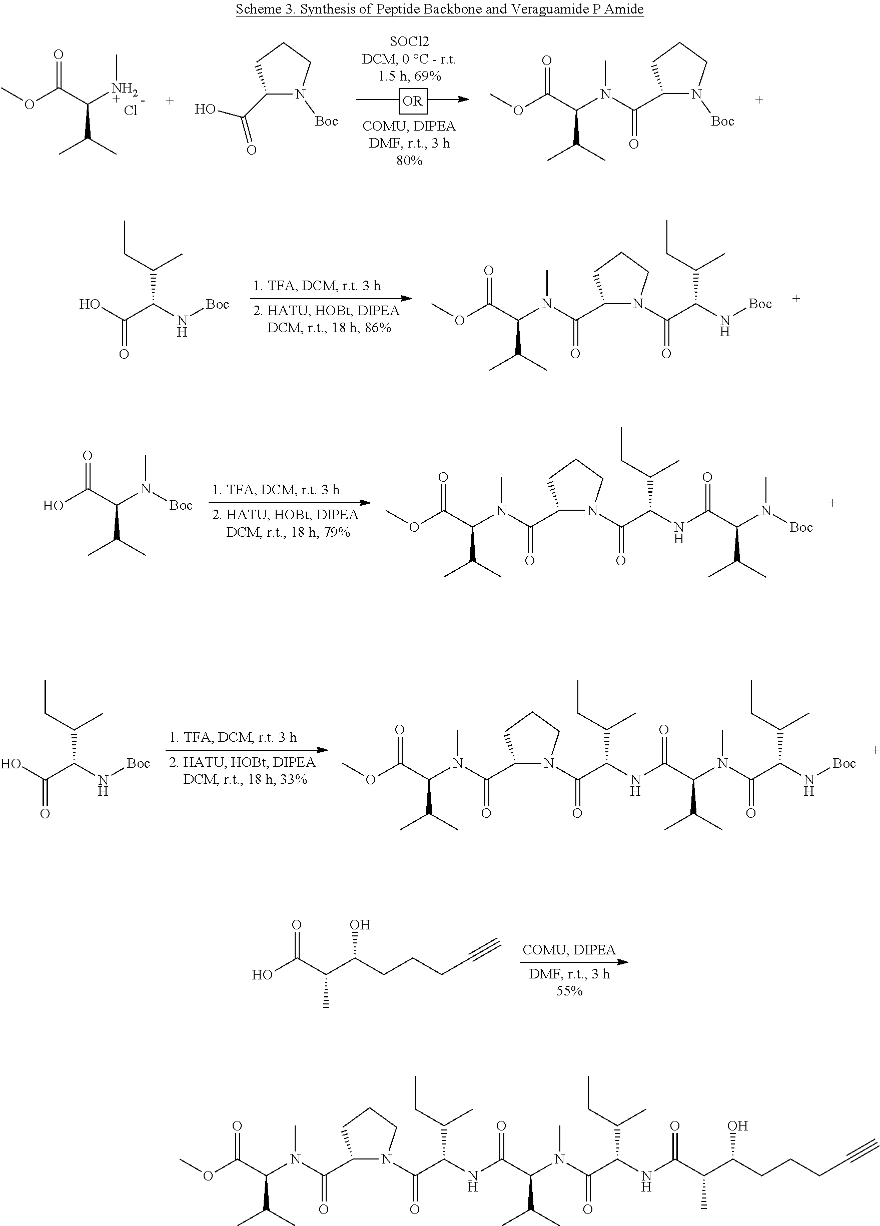

### Example 2. Cytotoxicity Testing of Veraguamide Analogs on HEK-293 Cells

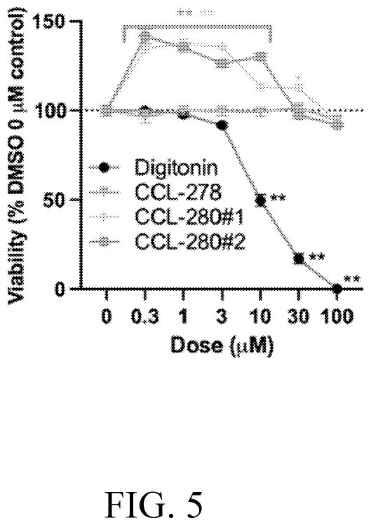

Several analogs were tested for cytotoxicity on HEK-293 cells in comparison to a positive cytotoxic control (digitonin) and the vehicle (3% by volume DMSO) (FIG. 5). The data was analyzed with a two-way ANOVA with Dunnett's test. As labeled in FIG. 5, * indicates p<0.05 and ** indicates p<0.01 versus a 0 μM dose.

The veraguamide analogs, CCL-228, CCL-280 #1, and CCL-280 #2 (chemical structures of the veraguamide analogs are illustrated in Scheme 4), were not cytotoxic. Some increase in activity was seen with CCL-280 #1 and CCL-280 #2. CCL-280 #1 and CCL-280 #2 are represented by the chemical structure of CCL-280 in Scheme 4 and were determined to be isolatable rotamers.

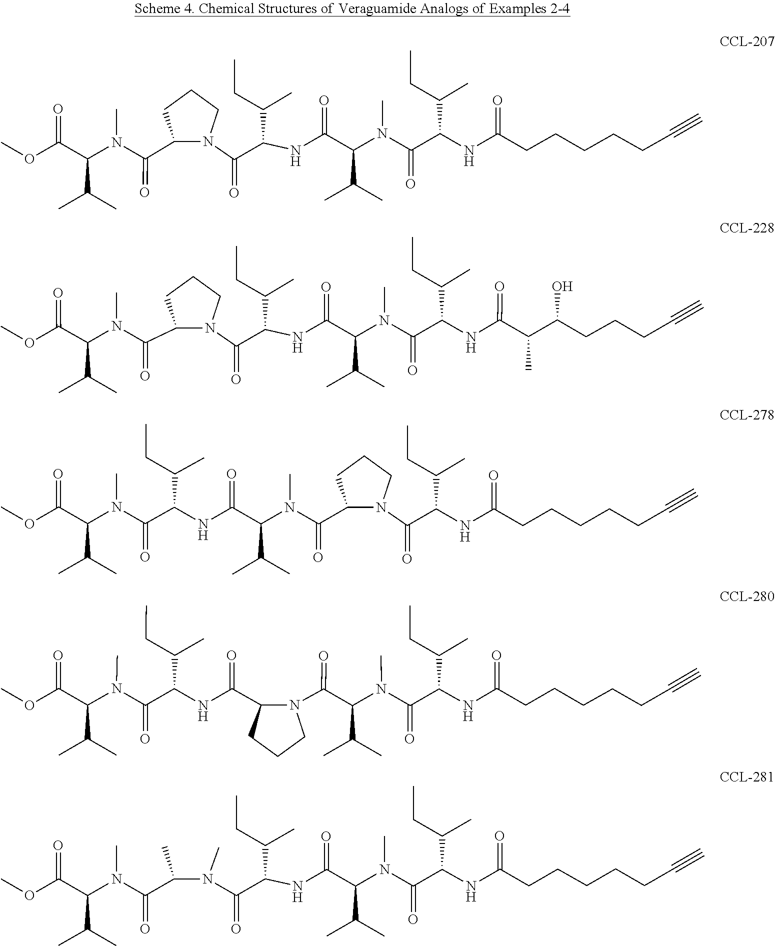

### Example 3. Inhibitory Potential in Human Nociceptor-Like Cells of Veraguamide Analogs

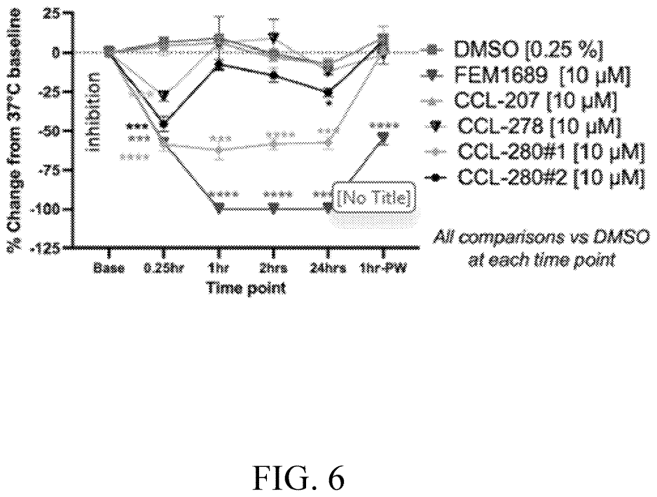

Several analogs (CCL-207, CCL-278, CCL-280 #1, and CCL-280 #2, see Scheme 4) were tested for inhibitory potential in human nociceptor-like cells. The analogs were compared to a positive control (FEM-1689) and a vehicle (0.25% by volume DMSO) (FIG. 6). The analogs were tested at 10 μM at multiple time points. As labeled in FIG. 6, the time point 1 hr-PW indicates an hour post washout. The data was analyzed with a two-way ANOVA with Dunnett's test. As labeled in FIG. 6, * indicates p<0.05 and ** indicates p<0.01, *** indicates p<0.001, and **** indicates p<0.0001 versus the DMSO control at the specified time point.

Several synthetic analogs (i.e., CCL-280 #1, CCL-280 #2, and CCL-278) demonstrated significant inhibitory (analgesic) effects (FIG. 6).

### Example 4. Effect of Veraguamide Analogs on the Integrated Stress Response

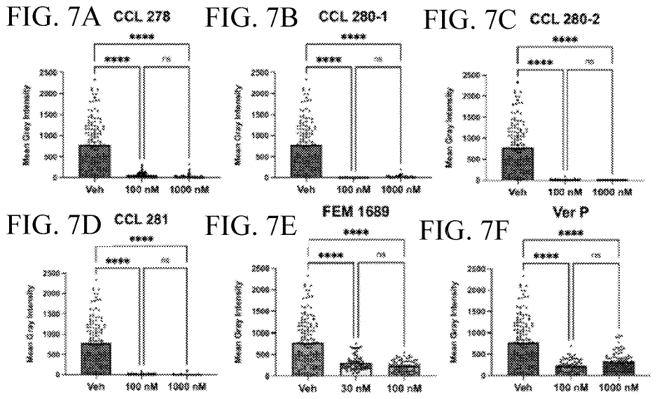

The Integrated Stress Response ISR has been shown to be active in nociceptors during chronic pain-like states in rodent models. Accordingly, veraguamide analogs (CCL-278, CCL-280-1, CCL-280-2, and CCL-281, see Scheme 4) were analyzed for reduction of phosphorylation of the eIF2α, a factor involved in ISR in mouse dorsal root ganglia (DRG). Both the parent natural compound (veraguamide P (2)) and the synthetic analogs inhibited the ISR consistently with an analgesic-like effect in comparison to the DMSO vehicle (FIGS. 7A to 7D and 7F). The veraguamide analogs exhibited significant analgesic-lief effects at 100 and 1000 nM concentrations. The effect was similar to that observed for the TMEM97 specific modulator FEM-1689 (FIG. 7E). The data was analyzed with a one-way ANOVA. As labeled in FIG. 7A to 7F, **** indicates p<0.00001.

All publications, patents, and patent applications mentioned in this specification are herein incorporated by reference to the same extent as if each individual publication, patent, or patent application was specifically and individually indicated to be incorporated by reference.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A veraguamide compound or pharmaceutically acceptable salt thereof, wherein the compound is derived from a veraguamide, wherein an ester linkage is replaced with an amide linkage.

2. The compound of claim 1, wherein the compound is a cyclic compound.

3. The compound of claim 1, wherein the compound is a linear compound.

4. The compound of claim 1 comprising one of the following compounds

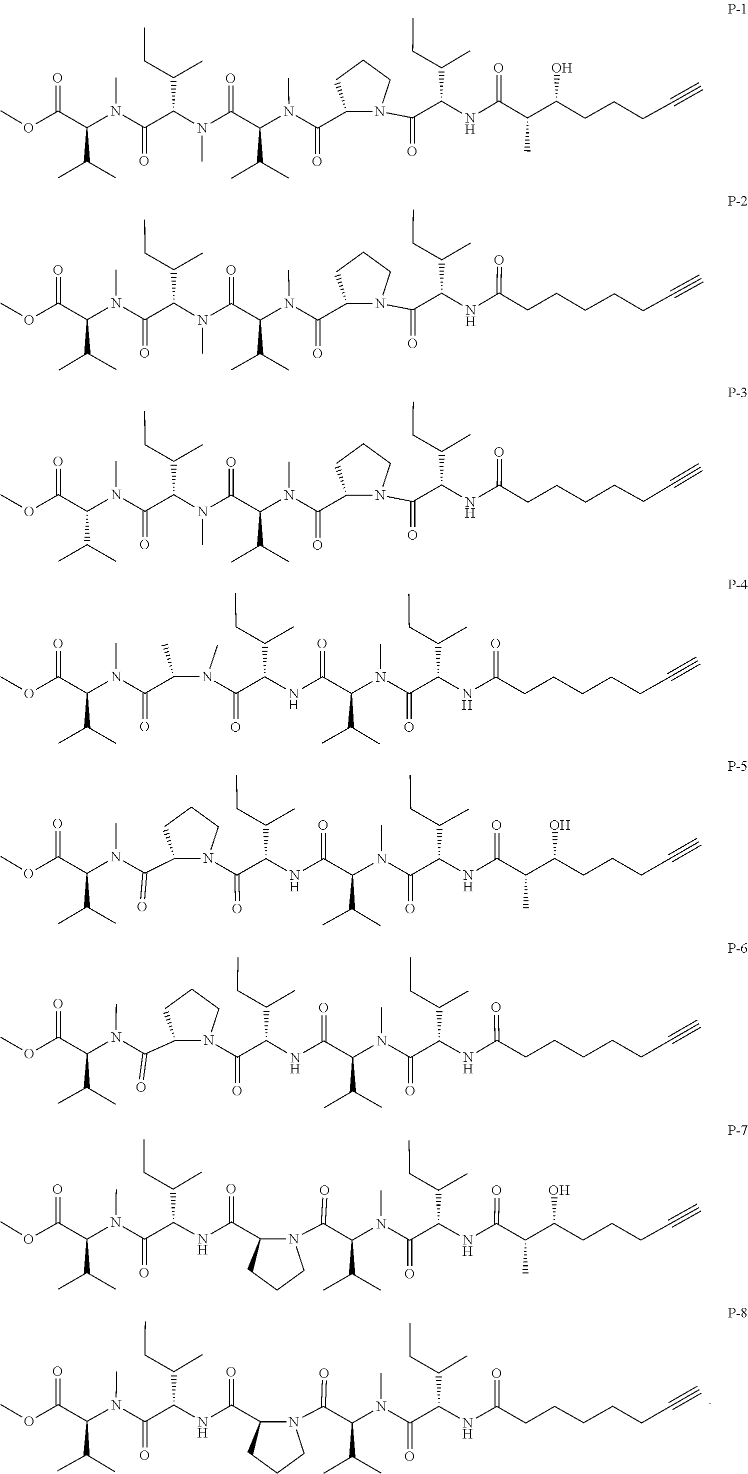

5. The compound of claim 1, wherein the terminal carboxylic acid group is replaced with a bioisosteric replacement for the carboxylic acid group.

6. The compound of claim 1, wherein the compound comprises 4 to 6 amino acid residues and a terminal group comprising an alkyne.

7. The compound of claim 1, wherein at least one amino acid residue is replaced with an unnatural amino acid.

8. The compound of claim 1, wherein the compound comprises a detectable group, preferably a substituted tetrazole group.

9. The compound of claim 1, wherein the compound comprises 4 to 6 amino acid residues represented by the following formula
-AA₁-AA₂-AA₃-AA₄-(AA₅)ₐ₅-(AA₆)ₐ₆,
wherein
a5 and a6 are each independently 0 or 1;
one or more of AA₁ to AA₆ is a proline;
the remainder of AA₁ to AA₆ that are not proline are proteinogenic amino acids, wherein the N of the amide linkages is optionally substituted.

10. A pharmaceutical composition comprising a compound of claim 1, a veraguamide natural product, or a combination thereof, and a pharmaceutically acceptable excipient.

11. The pharmaceutical composition of claim 10, further comprising an anesthetic adjuvant.

12. The pharmaceutical composition of claim 11, wherein the anesthetic adjuvant comprises a compound different from the compound of claim 1, a benzodiazepine, an alpha-2 agonist, an antiemetic, a steroid, or a combination thereof.

13. A method for treating or preventing pain in a patient in need thereof, comprising administering to the patient in need thereof a therapeutically effective amount of a compound of claim 1, a veraguamide natural product, or a combination thereof, or the pharmaceutical composition of claim 10.

15. A method for inhibiting a nociceptor cell, comprising contacting the cell with an effective amount of a compound of claim 1, a veraguamide natural product, or a combination thereof, or the pharmaceutical composition of claim 10.

16. A method for inhibiting phosphorylation of eukaryotic initiation factor 2α (eIF2α), comprising contacting the eukaryotic initiation factor 2× with an effective amount of a compound of claim 1, a veraguamide natural product, or a combination thereof, or the pharmaceutical composition of claim 10.
